(19) United States
(12) Patent Application Publication
(10) Pub. No.: US 2013/0267994 A1
(43) Pub. Date: Oct. 10, 2013

(54) Occlusion Attachment Device for a Tourniquet and Methods Associated Therewith

(71) Applicant: PHIL DURANGO LLC, Fraser, CO (US)

(72) Inventors: Tyler L. CROWDER, Folly Beach, SC (US); Dan CEDRONE, Huntersville, NC (US); Sarah A. CROWDER, Folly Beach, SC (US); Mark ESPOSITO, Fraser, CO (US)

(73) Assignee: PHIL DURANGO LLC, Fraser, CO (US)

(21) Appl. No.: 13/841,267

(22) Filed: Mar. 15, 2013

Related U.S. Application Data
(60) Provisional application No. 61/621,878, filed on Apr. 9, 2012.

(51) Int. Cl. A61B 17/132

(52) U.S. Cl. CPC A61B 17/1325
USPC 606/203

(57) ABSTRACT

An occlusion attachment device for coupling with a tourniquet includes a projection that can be attached using a platform to a portion of a tourniquet in an emergency situation, thereby providing a modified tourniquet that includes the projection. Clip-on, slide-on, and clamping structures associated with the platform are described.

### CROSS-REFERENCE TO RELATED APPLICATION

The present application claims the benefit of U.S. Provisional Patent Application No. 61/621,878 filed on Apr. 9, 2012, the entire contents of which are incorporated herein by reference in its entirety.

### FIELD

The present invention is related to a medical device, and more particularly to an occlusion attachment device for use with a tourniquet.

### BACKGROUND

Loss of blood is a major cause of death in emergency situations in which the injured person is alone or medical assistance is not immediately available. The use of a tourniquet to stop blood loss from an injured arm or leg is a well-known technique for preventing death in these situations.

Tourniquets operate by constricting the blood vessels within the limb to which it is applied, thereby stopping or occluding the flow of blood beyond the location of the tourniquet. If a tourniquet is used that includes a bulb or other device for focusing pressure on a portion of the patient's anatomy, then the bulb must be positioned correctly or its use can limit the actual effectiveness of the tourniquet. For typical soldiers, a regular tourniquet without a pressure focusing device is likely to be preferred over a more sophisticated device that requires special knowledge of the human anatomy, and in particular, the human vascular system. Accordingly, there is a need for a pressure focusing device that can be used with a typical field tourniquet if a person of specialized medical training is available to properly locate such pressure focusing device on the injured person.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of other embodiments.

At least one embodiment of the one or more inventions described herein is directed to an occlusion attachment device with a quick-connection fitting that can be coupled to a variety of different tourniquets, thereby providing a tourniquet with an occlusion attachment device coupled to the tourniquet.

In accordance with at least one embodiment, an occlusion attachment device for coupling to a tourniquet includes two oppositely facing surfaces for engaging a portion of the tourniquet, such as a base or a strap of the tourniquet. Accordingly, an occlusion attachment device for quickly coupling with a tourniquet is provided, the occlusion attachment device comprising: a bulbous projection attached to a platform, the bulbous projection having a tip located at least 0.5 inch from the platform, the platform further including two oppositely facing surfaces for engaging the tourniquet. In at least one embodiment, the two oppositely facing surfaces include a first surface and an oppositely facing second surface, wherein the first surface of the platform is associated with a first lateral side of the platform, wherein the oppositely facing second surface of the platform is associated with a second lateral side opposite the first lateral side of the platform. In at least one embodiment, the two oppositely facing surfaces include a first surface and an oppositely facing second surface, wherein the first surface of the platform is associated with a floor of the platform. In at least one embodiment, the oppositely facing second surface of the platform is associated with a level opposite the floor of the platform. In at least one embodiment, the level opposite the floor of the platform is interconnected to the floor of the platform by a lateral side of the platform. In at least one embodiment, the level opposite the floor of the platform is interconnected to the floor of the platform by a hinge. In at least one embodiment, the hinge is a living hinge having an expandable accordion shape. In at least one embodiment, the bulbous projection is adapted for attaching to the tourniquet without the use of any tools. In at least one embodiment, the bulbous projection comprises a shape selected from the group consisting of a portion of a sphere and a portion of an ellipsoid.

As described herein, several different types of structure are presented for a pressure focusing device that can be attached to a tourniquet. Accordingly, an occlusion attachment device for coupling with a tourniquet is provided, the occlusion attachment device comprising: a platform having a bulbous projection, wherein the platform includes means for quick-connection coupling to the tourniquet. In at least one embodiment, the bulbous projection is adapted for attaching to the tourniquet without the use of any tools. In at least one embodiment, the bulbous projection comprises a shape selected from the group consisting of a portion of a sphere and a portion of an ellipsoid.

The occlusion attachment devices described herein can be provided separately for use with various types of tourniquets. In addition, the occlusion attachment devices can be provided in an assembly, wherein the assembly includes both a tourniquet and an occlusion attachment device. Accordingly, an assembly for occluding blood flow is provided, comprising:
(a) a tourniquet comprising;
   a strap;
   a windlass operatively associated with the strap;
   a bendable base interconnected to the strap;
   a buckle interconnected to at least one of the strap and the bendable base; and
   means for securing the windlass from rotating; and
(b) an occlusion attachment device for coupling to the tourniquet, the occlusion attachment device including a bulbous projection, the occlusion attachment device not forming part of the tourniquet;
wherein the tourniquet is operable as a blood occluding device with or without association with the occlusion attachment device.

In at least one embodiment of the assembly, the occlusion attachment device includes a platform. In at least one embodiment of the assembly, the platform includes a first surface and an oppositely facing second surface, and wherein the first surface and the oppositely facing second surface are adapted for engaging a portion of the tourniquet. In at least one embodiment of the assembly, the portion of the tourniquet comprises one of the strap and the base. In at least one embodiment of the assembly, the first surface of the platform is associated with a first lateral side of the platform. In at least one embodiment of the assembly, the oppositely facing second surface of the platform is associated with a second lateral side opposite the first lateral side of the platform. In at least one embodiment of the assembly, the first surface of the platform is associated with a floor of the platform. In at least one embodiment of the assembly, the oppositely facing second surface of the platform is associated with a level opposite the floor of the platform. In at least one embodiment of the assembly, the first surface of the platform is associated with a first jaw member of the platform. In at least one embodiment of the assembly, the oppositely facing second surface of the platform is associated with a second jaw member opposite the first jaw member of the platform, and wherein the first jaw member is hingedly attached to the second jaw member. In at least one embodiment of the assembly, the occlusion attachment device is adapted for attaching to the tourniquet without the use of any tools. In at least one embodiment of the assembly, the bulbous projection comprises a shape selected from the group consisting of a portion of a sphere and a portion of an ellipsoid. In at least one embodiment of the assembly, the occlusion attachment device can slide longitudinally along the bendable base of the tourniquet after being attached to the base.

A further assembly is provided for occluding blood flow in a limb of a patient, comprising:

(a) a tourniquet comprising: a strap; a windlass operatively associated with the strap; a base interconnected to the strap and a buckle, the base including a first lateral side and a second lateral side; and

(b) an occlusion attachment device, wherein the occlusion attachment device is frictionally engageable with the lateral sides of the base.

In at least one embodiment, a portion of the base is inserted and/or pinched within the occlusion attachment device, and thereafter the tourniquet may be applied to the patient.

In accordance with at least one embodiment, a quick-connect occlusion attachment device is provided for coupling to a tourniquet without the use of tools, such as a flat head screwdriver. In accordance with at least one embodiment, a quick-connect occlusion attachment device is provided for coupling to a tourniquet wherein the occlusion attachment device is not threaded onto any portion of the tourniquet. In accordance with at least one embodiment, a quick-connect occlusion attachment device is provided wherein specific structure is not provided on the tourniquet to facilitate coupling the occlusion attachment device to the tourniquet; that is, the occlusion attachment device comprises structure for coupling to an existing tourniquet constructed without anticipating its use with an attachable occlusion attachment device.

In accordance with at least one embodiment, a tourniquet is provided that includes a removable pressure enhancement device. More particularly, a tourniquet can be manufactured and have associated with it a removable pressure enhancement device that includes a projection.

Various components are referred to herein as “operably associated.” As used herein, “operably associated” refers to components that are linked together in operable fashion, and encompasses embodiments in which components are linked directly, as well as embodiments in which additional components are placed between the two linked components.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the one or more present inventions, a more particular description of the one or more present inventions is rendered by reference to specific embodiments thereof which are illustrated in the appended drawings. It should be appreciated that these drawings depict only typical embodiments of the one or more present inventions and are therefore not to be considered limiting of its scope. The one or more present inventions are described and explained with additional specificity and detail through the use of the accompanying drawings in which:

FIG. 1 is a perspective view of a tourniquet known in the prior art, wherein the tourniquet is shown in use on a leg of a patient;

FIG. 2 is a top elevation view of the device shown in FIG. 1, wherein the device is stretched out along its longitudinal axis;

FIG. 3 is a side elevation of the device shown in FIG. 1, where the device is shown prior to tightening the device using a windlass;

FIG. 4 is a perspective view of a base of the device shown in FIG. 2;

FIG. 5A is a perspective view of an embodiment of an occlusion attachment device;

FIG. 5B is another perspective view of the occlusion attachment device shown in FIG. 5A;

FIG. 5C is a plan view of the occlusion attachment device shown in FIG. 5A;

FIG. 5D is an end elevation view of the occlusion attachment device shown in FIG. 5A;

FIG. 6A is a side perspective view of the occlusion attachment device of FIG. 5A being clipped on to a base of a tourniquet from a transverse orientation (wherein the strap and other structure of the tourniquet are omitted for clarity);

FIG. 6B is a side perspective view of the occlusion attachment device of FIG. 5A being clipped on to a base of a tourniquet using a rotation technique (wherein the strap and other structure of the tourniquet are omitted for clarity);

FIG. 6C is a side perspective view of the occlusion attachment device of FIG. 5A attached to a base of a tourniquet (wherein the strap and other structure of the tourniquet are omitted for clarity);

FIGS. 6D and 6E are perspective views of the occlusion attachment devices of FIGS. 5A and 7A attached to a base of a tourniquet;

FIG. 6F is a perspective view of the occlusion attachment devices of FIGS. 5A and 7A moved longitudinally right (relative to the drawing orientation) along the base;

FIG. 6G is a perspective view of the occlusion attachment devices of FIGS. 5A and 7A moved longitudinally left (relative to the drawing orientation) along the base;

FIG. 7A is a perspective view of another embodiment of an occlusion attachment device;

FIG. 7B is a perspective view of the occlusion attachment device shown in FIG. 7A;

FIG. 7C is perspective view of the occlusion attachment device shown in FIG. 7A;

FIG. 7D is a side elevation view of the occlusion attachment device shown in FIG. 7A;

FIG. 8A is a side perspective view of the occlusion attachment device of FIG. 7A being attached to a base of a tourniquet (wherein the strap and other structure of the tourniquet are omitted for clarity);

FIG. 8B is a side perspective view of the occlusion attachment device of FIG. 7A attached to a base of a tourniquet (wherein the strap and other structure of the tourniquet are omitted for clarity);

FIG. 8C is another side perspective view of the occlusion attachment device of FIG. 7A attached to a base of a tourniquet (wherein the strap and other structure of the tourniquet are omitted for clarity);

FIG. 9A is a perspective view of another embodiment of an occlusion attachment device, wherein the device is shown in an open position;

FIG. 9B is another perspective view of the occlusion attachment device shown in FIG. 9A, wherein the device is again shown in an open position;

FIG. 9C is another perspective view of the occlusion attachment device shown in FIG. 9A, wherein the device is shown in a closed position;

FIG. 9D is another perspective view of the occlusion attachment device shown in FIG. 9A, wherein the device is again shown in a closed position;

FIG. 10 is a perspective view of the occlusion attachment device of FIG. 9A attached to the strap of a tourniquet.

The drawings are not necessarily to scale.

### DETAILED DESCRIPTION

One or more embodiments of the one or more inventions described herein are directed to an occlusion attachment device for coupling with a tourniquet in an emergency situation. One or more embodiments may include an assembly that includes a tourniquet and a separate occlusion attachment device for optionally connecting to the tourniquet. For the embodiment described herein an occlusion attachment device that includes a projection can be attached to a portion of a tourniquet in an emergency situation, thereby providing a modified tourniquet that includes the projection. Before mating with the occlusion attachment device, the tourniquet is fully functional and can serve to treat a patient requiring a tourniquet. However, if desired, such as by a medically trained individual treating an injured person, an occlusion attachment device can be quickly attached to the tourniquet, thereby providing a tourniquet with an occlusion attachment device coupled to the tourniquet.

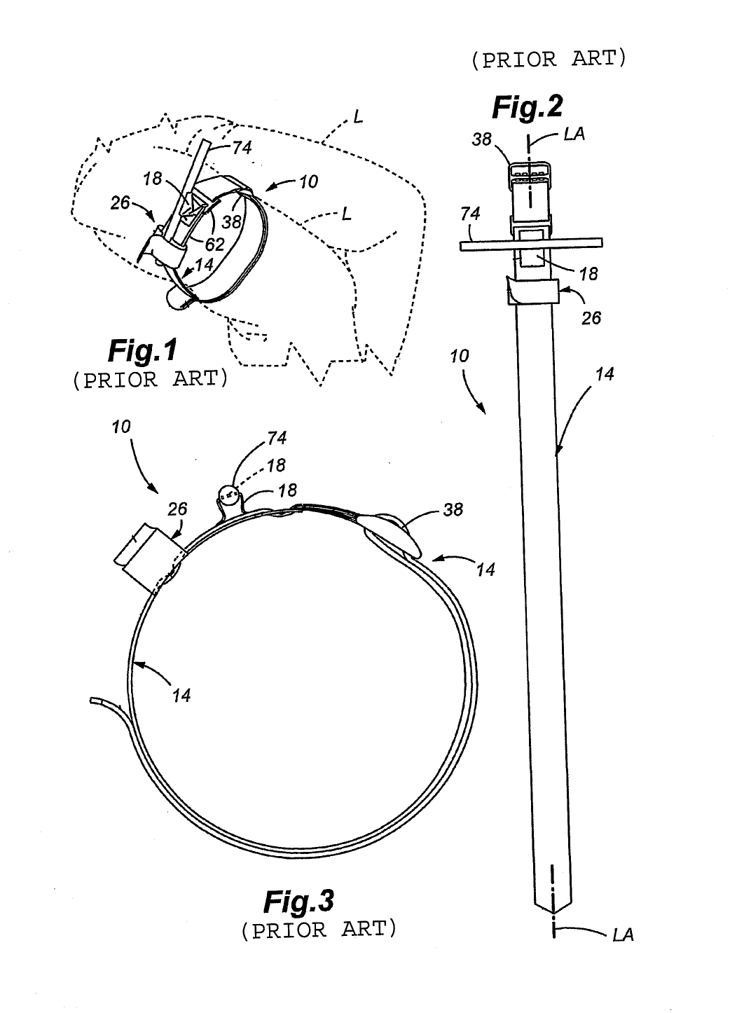

As noted above, in at least one embodiment an inventive assembly described herein includes a tourniquet and a separate occlusion attachment device with a quick-connection fitting for mating with the tourniquet. The tourniquet forming part of the assembly may be a variety of different types of tourniquets. By way of example, U.S. Pat. No. 7,892,253 (the content of which is incorporated herein by reference in its entirety) discloses a tourniquet that can be placed around a person's limb and tightened to stop the flow of blood in the person's limb beyond the location at which the tourniquet is placed. FIGS. 1-3 illustrate an embodiment of a tourniquet disclosed in U.S. Pat. No. 7,892,253. The tourniquet 10 includes an outer sleeve 14, an inner strap 18, a buckle 38, a tightening mechanism such as a windlass 74, a base 62 situated below the windlass, and a securing mechanism 26 to hold the windlass 74 after it has been rotated to tighten inner strap 18. The securing mechanism 26 may be a block, a curved or hooked member, an engageable or moveable ring or catch. The base 62 of the tourniquet 10 is located below the windlass 74 (that is, radially to the interior of the position of the windlass 74 when positioned on a patient's limb). In addition, a bottom of the base 62 is located adjacent the patient's limb when positioned on the patient's limb.

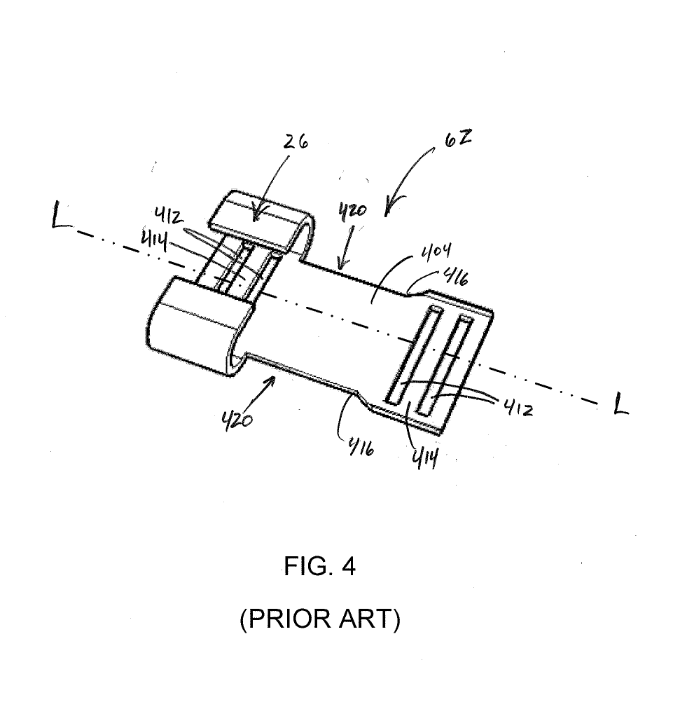

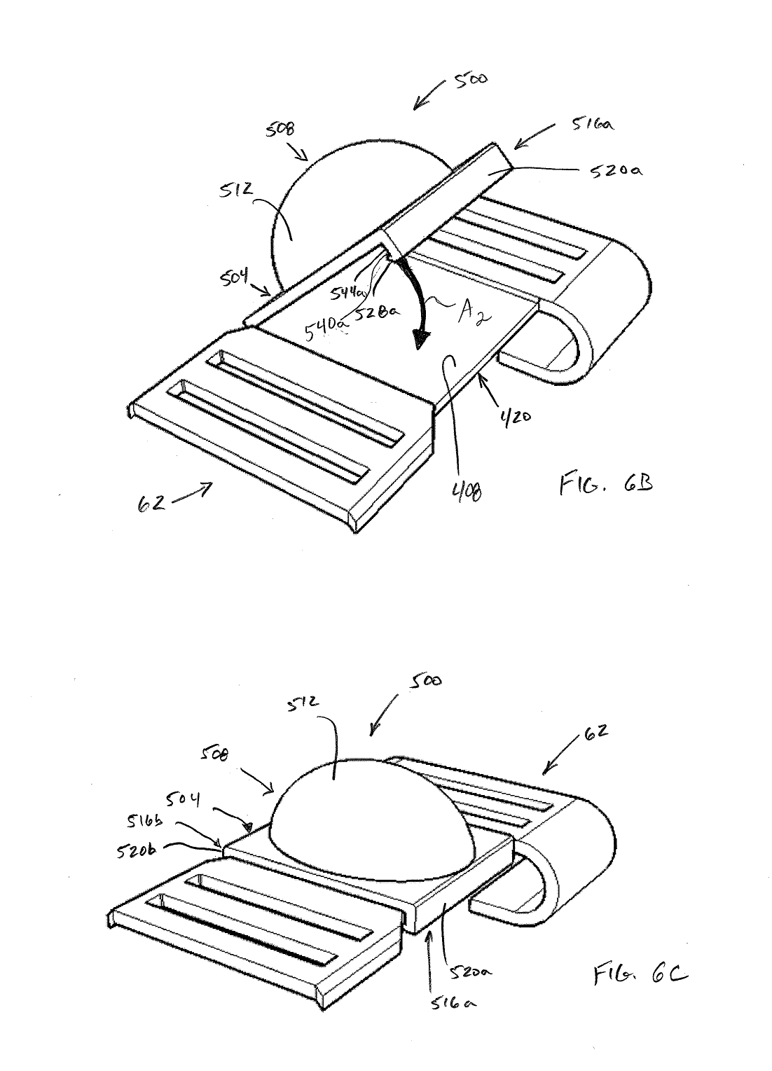

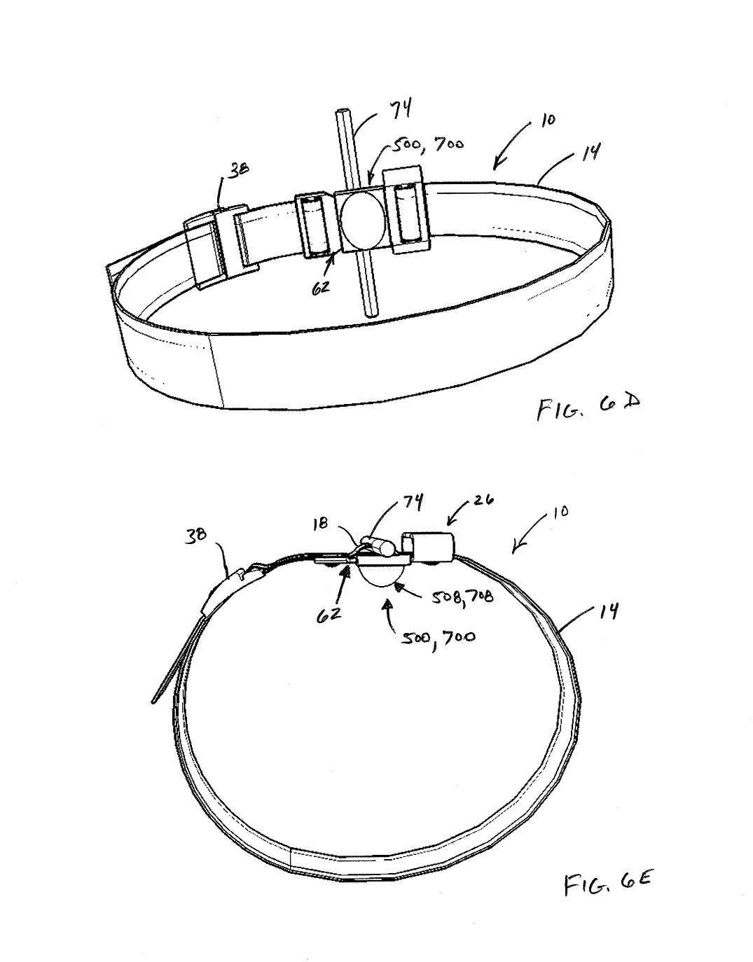

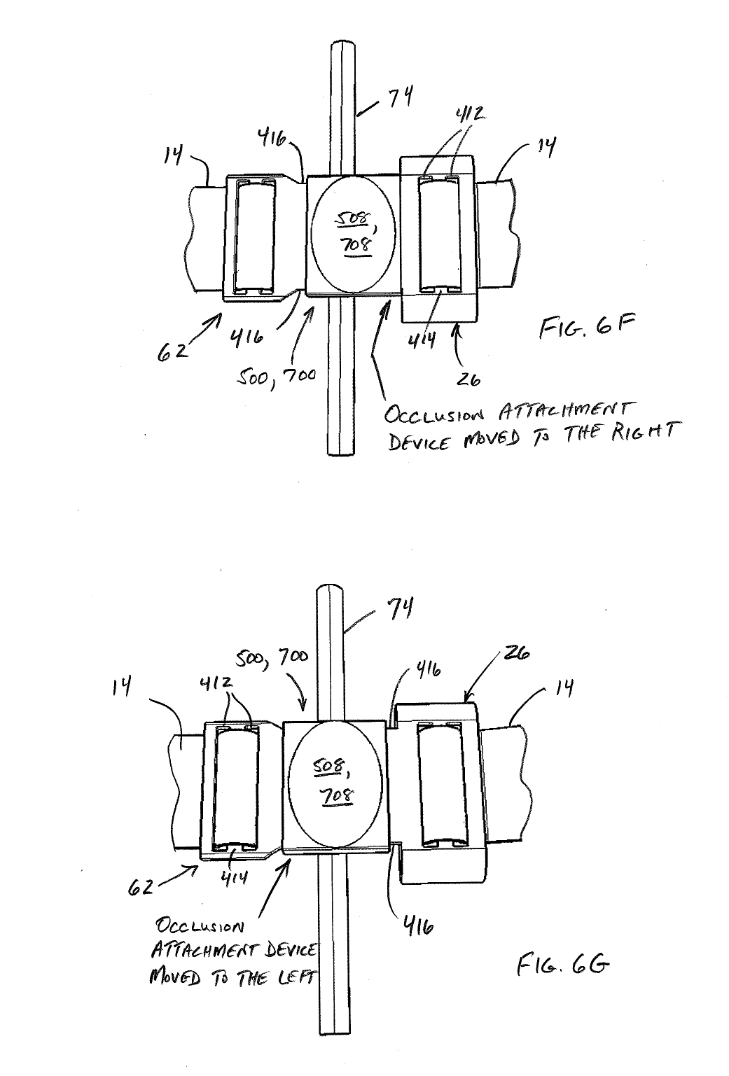

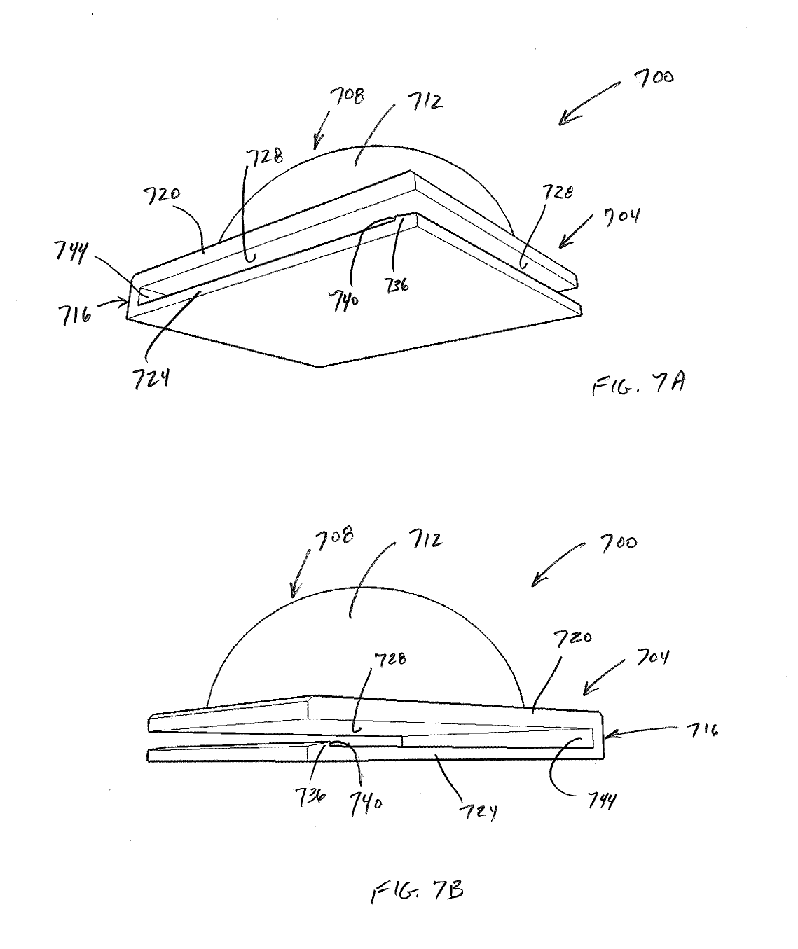

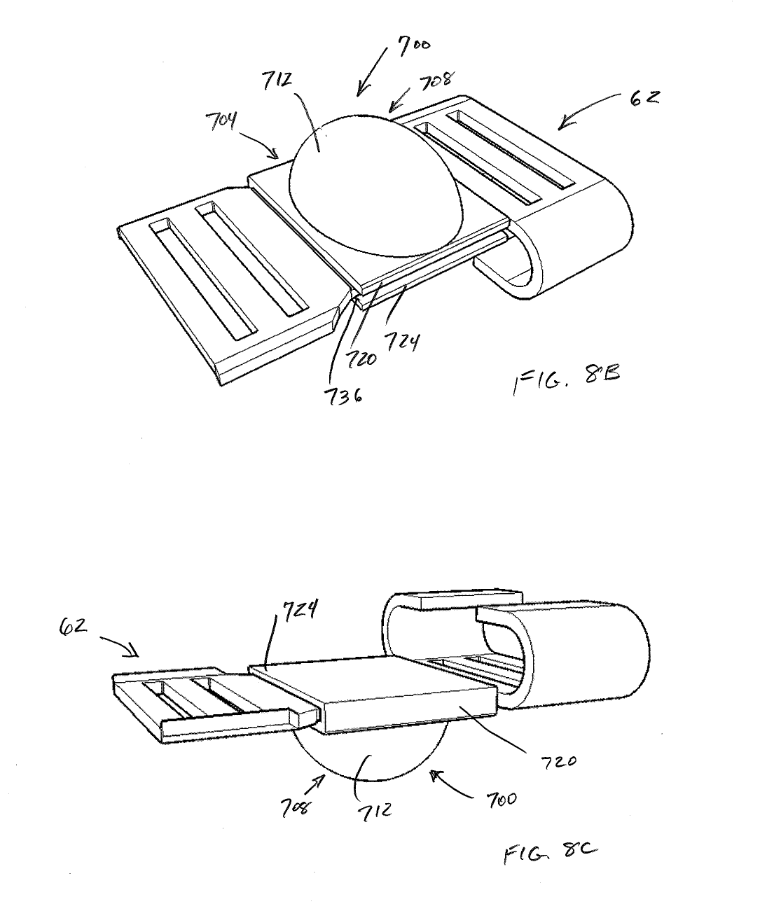

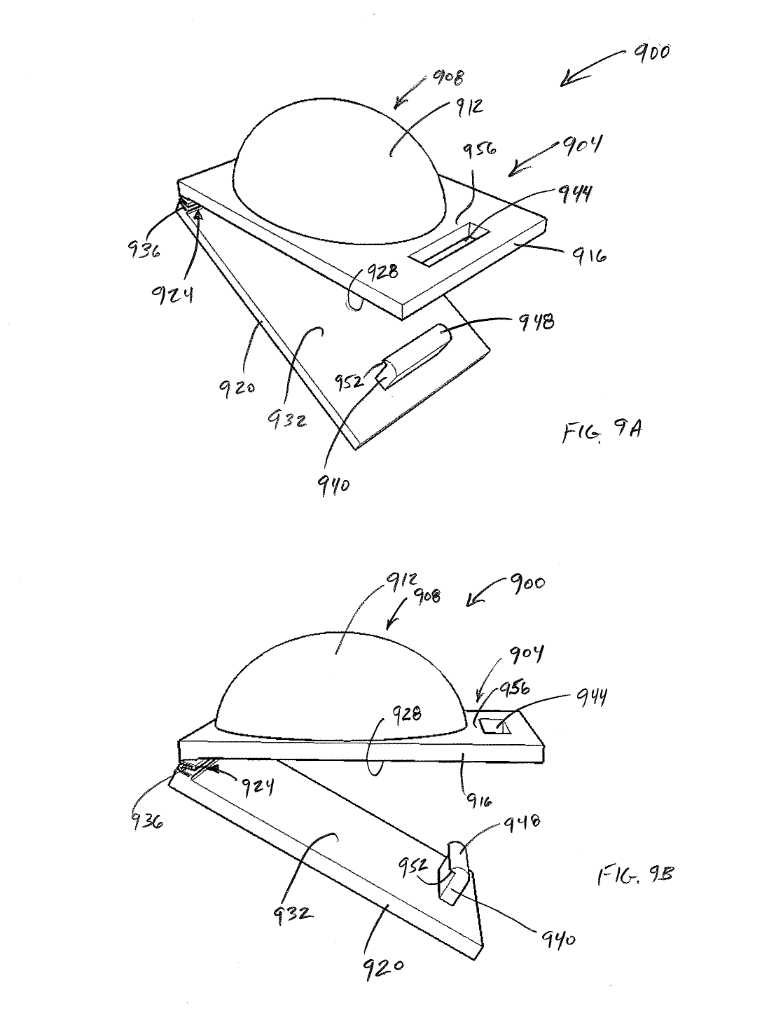

Referring now to FIG. 4, a more detail view is shown of a base 62 that may be used with a tourniquet, such as tourniquet 10 described in U.S. Pat. No. 7,892,253. The base 62 may be formed of thermoplastic or moldable (as for example, injection molded) plastic type of material. The base 62 includes an upper surface or top surface 404 that faces the windlass 74. The base 62 further includes a lower surface or bottom surface 408 (as best seen in FIGS. 6A and 8A). The base 62 may also include one or more structures for engaging at least one of a sleeve 14 or strap 18 associated with the tourniquet 10, such as first and second openings 412 and weaving bars 414 at opposite longitudinal ends of the base 62 for receiving at least a portion of at least one of a sleeve 14 or strap 18 associated with the tourniquet 10. In addition, and as illustrated in FIGS. 1-4, the base 62 may include a securing mechanism 26 for limiting unwinding of the windlass 74 after the windlass 74 is used to tighten the inner strap 18 associated with the tourniquet 10. In at least one embodiment, the base 62 is not simply rectangular in shape, but includes lateral recesses 416 along its lateral sides 420. FIG. 4 also shows the longitudinal axis L-L of the base 62, wherein the longitudinal axis L-L of the base 62 substantially corresponds to a longitudinal axis of the tourniquet running down the length of the tourniquet to which the base 62 is associated. As will be appreciated by those skilled in the art, other tourniquets having a base may not include the structure shown in FIG. 4, but may still share top and bottom surfaces and may be suitable for receiving an embodiment of the occlusion attachment devices described herein.

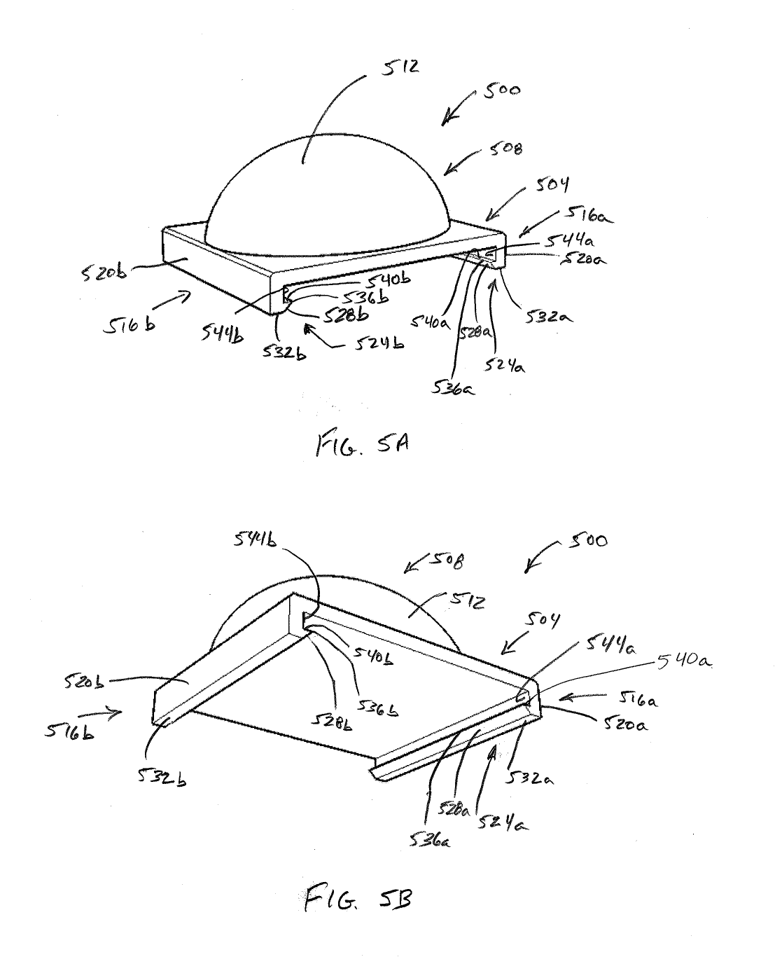

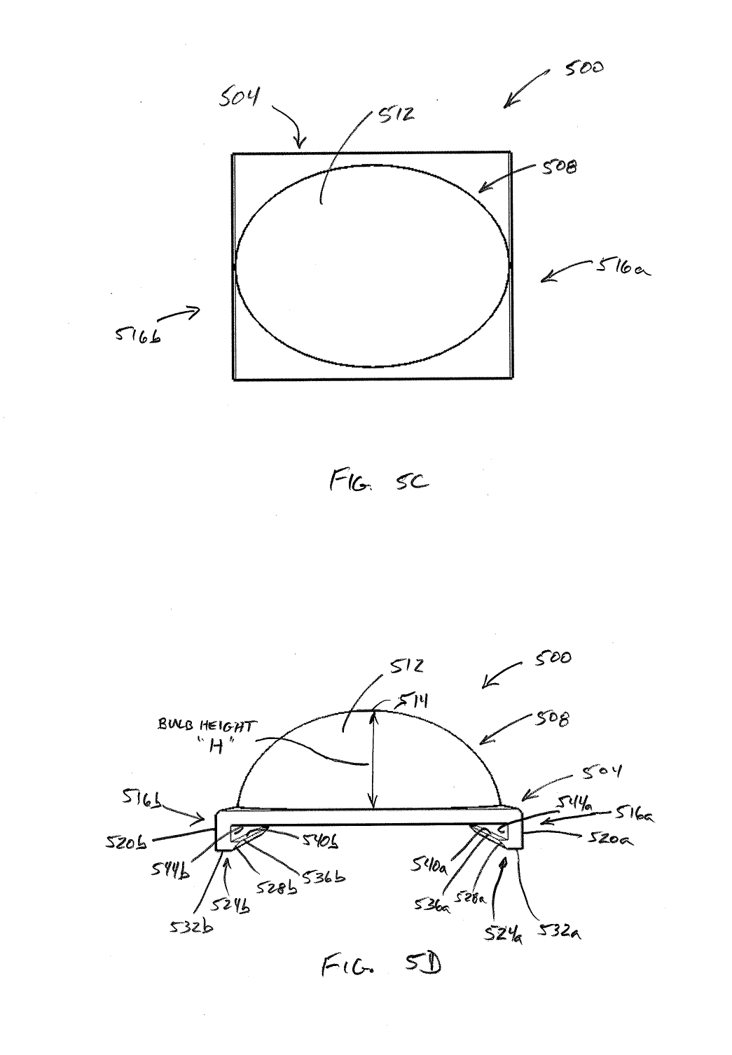

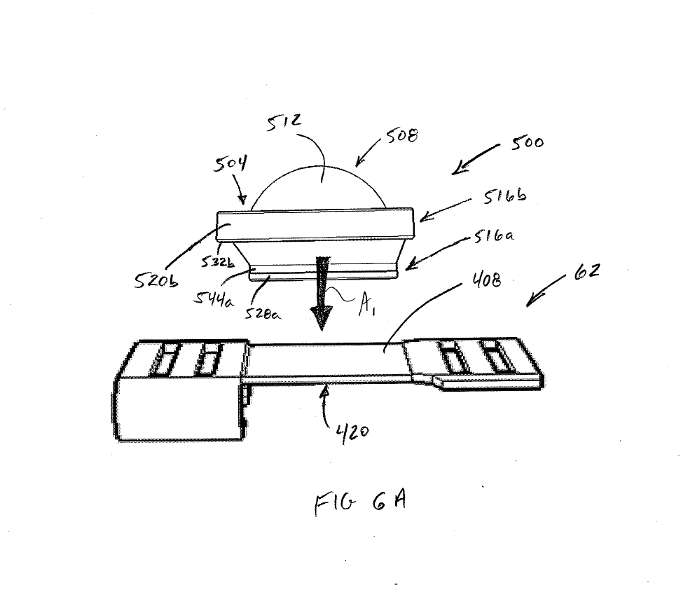

Referring now to FIGS. 5A-5D, an embodiment of an occlusion attachment device is shown. Occlusion attachment device 500 includes a platform 504 and a bulbous portion, protrusion or projection 508 for providing a focused pressure to assist in occluding blood flow when used in combination with a tourniquet, such as tourniquet 10. As discussed further below, the projection 508 preferably includes a rounded or curved surface 512 for contacting the patient's skin or a material (e.g., shirt or pants) overlying the patient's skin. The projection has a tip 514, wherein the tip 514 is preferably at least about 0.5 inch from the adjacent platform surface, so that the projection has a bulb height “H” suitable for assisting with occluding blood flow associated with the vascular system of the patient.

Occlusion attachment device 500 further includes a mating structure for coupling with an existing tourniquet. As depicted in FIGS. 5A-5D, platform 504 includes lateral sides 516a and 516b. In at least one embodiment, the lateral sides 516a and 516b of the platform 504 each include a side panel 520a and 520b, respectively, with inwardly situated ledges 524a and 524b. The ledges 524a and 524b preferably comprise sloped surfaces 528a and 528b grading from a bottom 532a and 532b of the ledges 524a and 524b to an inward edge 536a and 536b adjacent respective coupling shoulder surfaces 540a and 540b. The coupling shoulder surfaces 540a and 540b are adapted for engaging the top surface 404 of the base 62 of the tourniquet. Each lateral side or side panel 520a and 520b includes an interior side panel surface 544a and 544b, wherein when the occlusion attachment device 500 is coupled to the tourniquet, the interior side panel surfaces 544a and 544b form oppositely facing surfaces that engage the lateral sides 420 of a base of a tourniquet, such as base 62 of tourniquet 10.

To couple the occlusion attachment device 500 with a tourniquet, and with reference now to FIG. 6A, the occlusion attachment device 500 is positioned adjacent the underside of the base 62 of the tourniquet and then coupled to the base 62. For example, the occlusion attachment device 500 may be quickly attached to the base 62 of the tourniquet by pressing the occlusion attachment device 500, per arrow A₁, against the bottom of the base 62 of a tourniquet so that the occlusion attachment device 500 clips on to the base 62 of the tourniquet, such as is shown in FIG. 6C. As those skilled in art will appreciate, the sloped surfaces 528a, 528b of the ledges 524a, 524b ease coupling the occlusion attachment device 500 to the tourniquet's base 62.

Alternatively, the occlusion attachment device 500 may coupled to the base 62 of a tourniquet by rotating the occlusion attachment device 500, per arrow A₂, into coupling engagement with the base 62. More particularly, and with reference now to FIG. 6B, a first lateral side 516a may be positioned to engage a portion of the base 62 of a tourniquet. Thereafter the occlusion attachment device 500 can be rotated, per arrow A₂, or otherwise pressed to engage the base 62 of the tourniquet until the occlusion attachment device 500 clips on to the base 62 of the tourniquet, such as is shown in FIG. 6C. Whether pressed or rotated into coupling engagement, the lateral sides 516a, 516b of the occlusion attachment device 500 are situated to engage the lateral sides 420 of the base 62, and after being pressed into a coupling engagement with the base, the coupling shoulder surfaces 540a and 540b of the occlusion attachment device 500 are located in apposing engagement with the top surface 404 of the base. Here it is noted that another material may reside along the top surface 404 of the base 62 of a tourniquet. By way of example, for tourniquet 10 described above, a layer of material associated with the tourniquet's outer sleeve 14 may reside along the top surface 404 of the base 62 between the windlass 74 and the top surface 404 of the base 62. Nonetheless, the advantageous configuration of the occlusion attachment device 500 allows the lateral sides 516a and 516b, including the coupling shoulder surfaces 540a and 540b, as well as the interior side panel surfaces 544a and 544b, to mate the occlusion attachment device 500 to the base 62 of the tourniquet regardless of whether or not the tourniquet possesses a layer of outer sleeve material adjacent the top surface of the base.

Referring now to FIGS. 6D and 6E, occlusion attachment device 500 is shown after having been clipped on to a base of a tourniquet, such as base 62 of tourniquet 10. The projection 508 of the occlusion attachment device 500 is located radially to the interior of the base of the tourniquet.

Referring now to FIGS. 6F and 6G, the occlusion attachment device 500, is preferably properly sized for a given base of a tourniquet to allow a minor amount of longitudinal travel on the base 62 after the occlusion attachment device 500 is mounted to the tourniquet. More particularly, by permitting a minor amount of longitudinal travel in the event that the tourniquet rotates around the extremity as the tourniquet is tightened, this minor amount of longitudinal travel advantageously permits the occlusion attachment device 500 to remain over the artery to which is applied, thereby providing effective occlusive pressure. As shown in FIG. 6F, the occlusion attachment device can travel to a right-most portion of the recess 416 of the base 62. As shown in FIG. 6G, the occlusion attachment device can travel to a left-most portion of the recess 416 of the base 62. Thus, in use, the operator of the tourniquet positions the occlusion attachment device 500 over the targeted artery when initiating tightening of the tourniquet. Thereafter, and because the occlusion attachment device 500 is in a longitudinal slidable engagement with the base 62 of the tourniquet, if the tourniquet rotates around the extremity as it is tightened, the occlusion attachment device will remain positioned over the artery providing effective occlusive pressure.

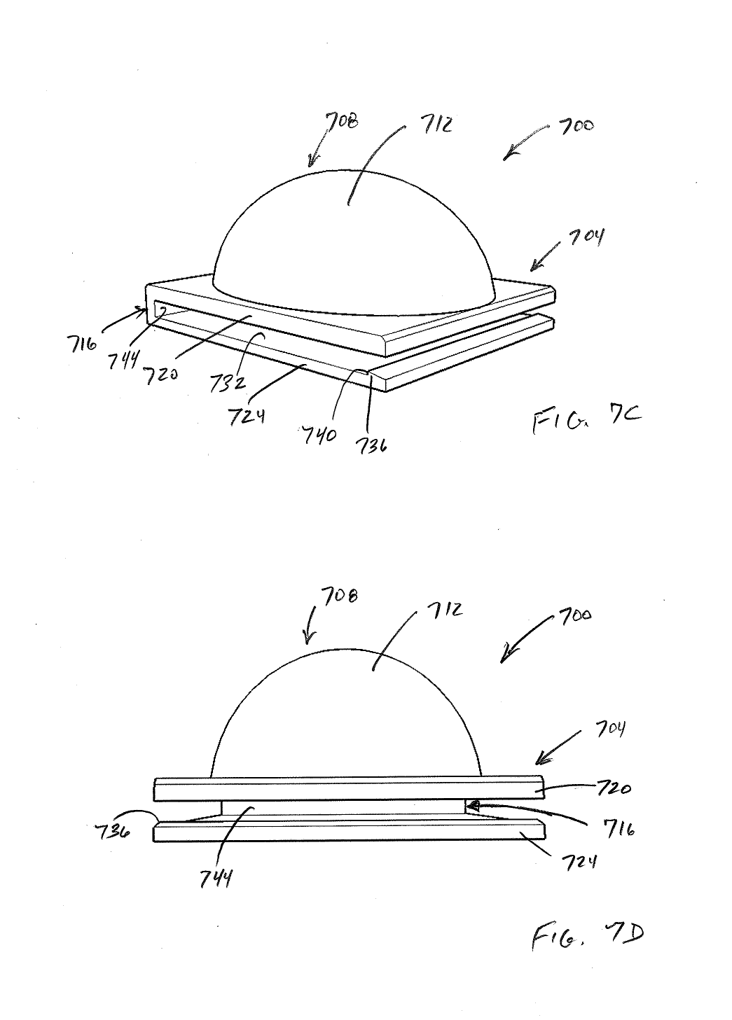

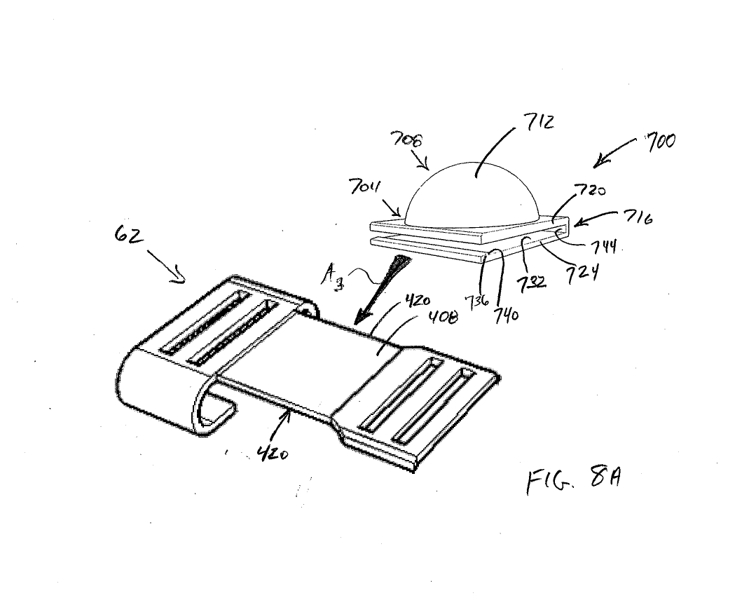

Referring now to FIGS. 7A-7D, and in accordance with at least one embodiment, another occlusion attachment device is shown. Occlusion attachment device 700 is configured with structure to slidably engage a portion of the base for coupling to the base 62 of a tourniquet. Occlusion attachment device 700 includes a platform 704 and a protrusion or projection 708 for providing a focused pressure to assist in occluding blood flow. As with projection 508 of occlusion attachment device 500, the projection 708 of occlusion attachment device 700 preferably includes a rounded or curved surface 712 for contacting the patient's skin or a material (e.g., shirt or pants) overlying the patient's skin. Occlusion attachment device 700 further includes a way of coupling with an existing tourniquet. More particularly, the platform 704 preferably includes three open sides with a single lateral side 716 and with a platform floor 720 spaced apart from a second level 724. An interior floor surface 728 of the platform floor 720 is located opposite an interior second level surface 732 of the second level 724. In at least one embodiment, the second level 724 is biased toward the platform floor 720 so as to create a pinching force to hold the tourniquet base once the occlusion attachment device 700 is pushed onto the base 62 of the tourniquet.

In at least one embodiment, at least one of the platform floor 720 or the second level 724 include a lip 736 with an inward facing lip support surface 740 for engaging a lateral side 420 of a base 62 of a tourniquet. The lip support surface 740 is located opposite an interior lateral side surface 744 of the lateral side 716. Once the occlusion attachment device 700 is pushed onto the base of the tourniquet, interior lateral side surface 744 and the inward facing lip support surface 740 assist in holding the lateral sides 420 of the tourniquet's base 62 within the occlusion attachment device 700.

To couple the occlusion attachment device 700 with a tourniquet, and with reference now to FIG. 8A, the occlusion attachment device 700 is positioned adjacent one of the lateral sides 420 of the base 62 of the tourniquet and is then pushed on to the base 62, per arrow A₃. For example, the occlusion attachment device 700 may be quickly attached to the tourniquet by inserting the base 62 into the opening of the occlusion attachment device 700 that resides between the platform floor 720 and the second level 724. Once the base of the tourniquet is inserted or slid into the opening of the occlusion attachment device 700, the base of the tourniquet is positioned between the interior floor surface 728 of the platform floor 720 and the interior second level surface 732 of the second level 724, such as is shown in FIGS. 8B and 8C.

Referring again back to FIGS. 6D and 6E, occlusion attachment device 700 is shown after having been clipped on to a base of a tourniquet, such as base 62 of tourniquet 10. The projection 708 of the occlusion attachment device 700 is located radially to the interior of the base of the tourniquet.

Referring again back to FIGS. 6F and 6G, and as noted above in the discussion regarding occlusion attachment device 500, the occlusion attachment device 700 is also preferably sized to allow a minor amount of longitudinal travel along the base 62 of a tourniquet as the tourniquet is tightened. This feature advantageously permits the occlusion attachment device 700 to remain over the artery to which it is applied, thereby providing effective occlusive pressure.

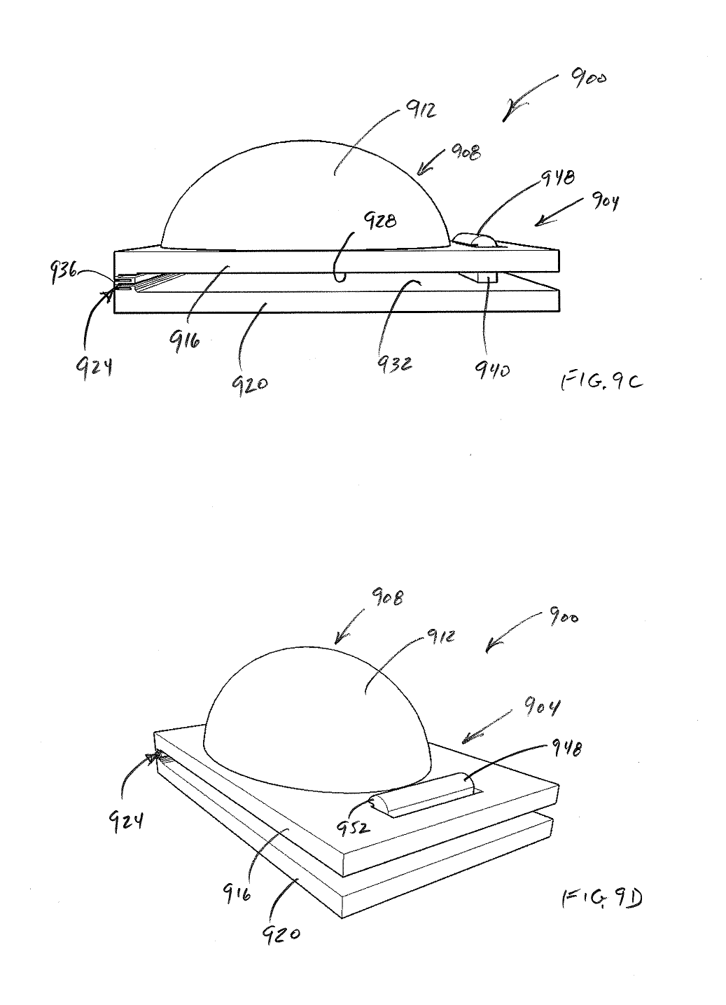

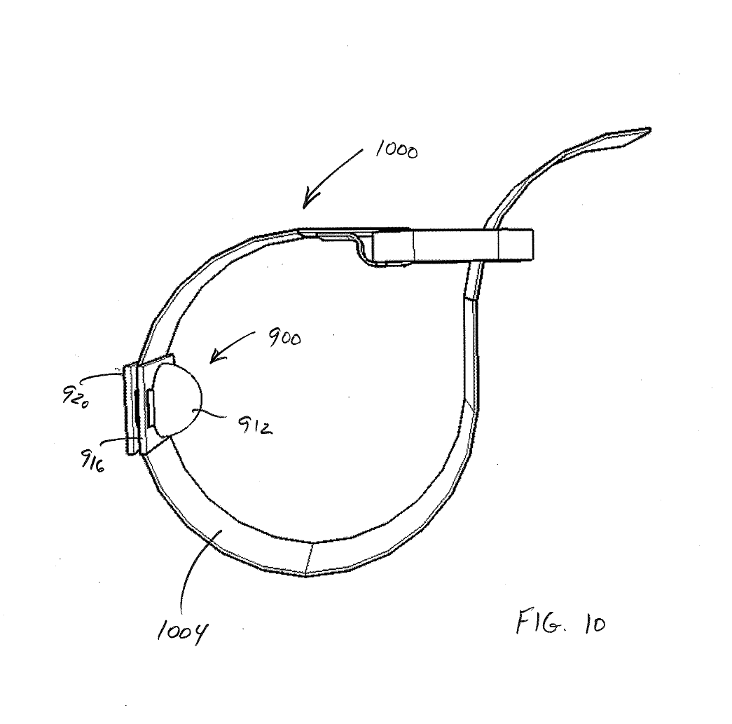

Referring now to FIGS. 9A-D, and in accordance with at least one embodiment, another occlusion attachment device is shown. Occlusion attachment device 900 is configured with structure to clamp to a portion of a tourniquet, such as strap of the tourniquet. Occlusion attachment device 900 is suitable for coupling with a number of types of tourniquets, including the tourniquet described in U.S. Pat. No. 7,892,253.

Occlusion attachment device 900 includes a platform 904 and a protrusion or projection 908 for providing a focused pressure to assist in occluding blood flow. As with projections 508 and 708 of occlusion attachment devices 500 and 700 respectively, the projection 908 of occlusion attachment device 900 preferably includes a rounded or curved surface 912 for contacting the patient's skin or a material (e.g., shirt or pants) overlying the patient's skin. Occlusion attachment device 900 further includes a way of coupling with an existing tourniquet. More particularly, the platform 904 preferably includes a portion that articulates, such as first jaw member 916 that is able to move relative to second jaw member 920. In at least one embodiment, the first jaw member 916 is hingedly attached to the second jaw member 920, such as by a pin or living hinge 924. An interior first jaw member surface 928 of the first jaw member 916 is located opposite an interior second jaw member surface 932 of the second jaw member 920.

Advantageously, in at least one embodiment a living hinge 924 includes an accordion structure 936 (as best seen in FIG. 9C). The accordion structure 936 allows the clamping action of occlusion attachment device 900 to accommodate tourniquet structure of different thicknesses, such as tourniquets that have relatively thin straps or relatively thick straps. Although described herein as coupling with a strap of a tourniquet, it is to be understood that the occlusion attachment device 900 may be used to couple with other tourniquet structures, such as a base should the base and the occlusion attachment device be compatible.

In accordance with at least one embodiment, the platform 904 includes structure for securing the first jaw member 916 to the second jaw member 920 to hold the occlusion attachment device 900 to the tourniquet. More particularly, a latch mechanism may be employed to secure the first jaw member 916 to the second jaw member 920. By way of example and not limitation, a latch mechanism comprising a male member 940 may be coupled with a female opening 944, wherein one of the first and second jaw members 916, 920 includes the male member 940 and the other of the first and second jaw members 916, 920 includes the female opening 944. FIGS. 9A-9D illustrate a possible configuration wherein the first jaw member 916 includes the female opening 944 and the second jaw member 920 includes the male member 940. In at least one embodiment, the male member 940 preferably includes a rounded distal end 948 with a lateral extension 952 for engaging a platform surface 956 adjacent the female opening 944.

To couple the occlusion attachment device 900 with a tourniquet 1000, and with reference now to FIG. 10, the occlusion attachment device 900 is opened by separating the first jaw member 916 from the second jaw member 920, and then the strap 1004 of the tourniquet is inserted within the first and second jaw members 916, 920 followed by closing the first and second jaw member 916, 920 such that the latching mechanism engages. The tourniquet 1000 may then be applied to a patient with the occlusion attachment device 900 positioned as desired by the person applying the tourniquet.

In accordance with at least one embodiment, a pressure inducing bulb is generally bulbous in shape, or is similar in shape to a half sphere, a portion of a sphere, or a portion of an ellipsoid.

In accordance with at least one embodiment, multiple size protrusion bulbs may be available for use with a given tourniquet. More particularly, a single tourniquet may be coupled with a first attachable protrusion bulb of a first size, or alternatively, the same tourniquet may be coupled with a second attachable protrusion bulb of a second size. Different size protrusion bulbs may be provided for accommodating arms as opposed to legs, and/or for treating a smaller person, such as a child, as opposed to a large person, such as a large man. For the various embodiments presented herein, the protrusion bulbs have a bulb height “H,” as best seen in FIG. 5D, of at least about 0.5 inch.

Embodiments of the occlusion attachment devices described herein may be made out of a material suitable for its intended purpose. By way of example, the occlusion attachment devices described herein may be manufactured from one or more man-made materials. As a further non-limiting example, nylon can be used to make the occlusion attachment devices described herein. Alternatively, other materials may be used, such as urethane (e.g., cast urethane made by pouring urethane into a mold). The occlusion attachment devices can be machined or made by injection molding techniques, or otherwise made by another suitable process. For example, the occlusion attachment devices described herein can be made using ABS plastic using stereolithography (a type of 3D printing).

In addition to the structure described above, other means for attaching a pressure inducing bulb or occlusion attachment device may be used. In accordance with at least one embodiment, an occlusion attachment device without a platform may be used. By way of example, an occlusion attachment device without a platform may be used wherein a surface of the occlusion attachment device, such as a substantially planar surface of the pressure inducing bulb, is attached to a portion of a tourniquet, such as at a bottom surface of the base of the tourniquet.

In at least one embodiment, a hook and loop fastener is used to attach the pressure inducing bulb to the bottom surface of the tourniquet's base. Once the hook and loop fastener of the optional pressure enhancing device or a pressure inducing bulb is coupled to the mating surface of the base of the tourniquet, the tourniquet may be applied to the patient's limb.

In a least one embodiment, a hook portion of a hook and loop fastener is associated with an optional pressure enhancing device and a loop portion of a hook and loop fastener is located on a bottom surface of the base of the tourniquet. Such configuration allows the softer loop material to be exposed on the underside of the base for instances wherein the optional pressure enhancing device is not coupled to the tourniquet before the tourniquet is applied to a limb of a patient.

In accordance with several embodiments described herein, the mechanism for fastening the optional pressure enhancing device to a tourniquet is not necessarily permanent. By way of example, at least the clip-on, slide-on and use of hook and loop fastener materials allow for removal of the optional pressure enhancing device if the use of the optional pressure enhancing device with the tourniquet is later not desired.

In addition to the structure described above, still other means may be used to couple an optional pressure enhancing device or a pressure inducing bulb to a tourniquet. By way of example, a male/female fitting such as one or more prongs may be situated on a platform or surface of the optional pressure enhancing device or a pressure inducing bulb for insertion into receptacles located within the base of the tourniquet. Once the one or more prongs are inserted into the receptacles of the base of the tourniquet, the tourniquet may be applied to the patient's limb.

In accordance with at least one embodiment, a tourniquet is provided that includes a removable pressure enhancement device. More particularly, a tourniquet can be manufactured and have associated with it a removable pressure enhancement device that includes a projection. If desired by the operator of the tourniquet, the pressure enhancement device can be removed from the tourniquet, and then the tourniquet applied without the pressure enhancement device. So, for example, a tourniquet may include one of the occlusion attachment devices 500, 700, 900 as described herein. Upon initially accessing and/or intending to use the tourniquet, such as to treat a wounded person, the occlusion attachment device can be removed. By way of example, the occlusion attachment device 500 can be unclipped from the tourniquet shown in FIGS. 6D and 6E by grasping the platform 504 of the occlusion attachment device 500 and removing it from base 62. By way of further example, the occlusion attachment device 700 can be slid off from the tourniquet shown in FIGS. 6D and 6E by grasping the platform 704 of the occlusion attachment device 700 and removing it from base 62. In addition, the occlusion attachment device 900 can be unclamped from the tourniquet shown in FIG. 10 by releasing the jaws of occlusion attachment device 900 and removing it from strap 1004. Of course, the operator may optionally decide to use the tourniquet with the removable occlusion attachment device still remaining on the tourniquet.

Accordingly, in at least one embodiment, a tourniquet is provided, the tourniquet comprising: a strap; a windlass engaged and operatively associated with the strap; a base interconnected to the strap, the base including an upper surface facing the windlass, a bottom surface, and a plurality of sides and edges; a buckle interconnected to at least one of the strap and the base; and a removable pressure enhancement device attached to at least one of the base and the strap. In at least one embodiment, the removable pressure enhancement device is frictionally engaged with the base. In at least one embodiment, the removable pressure enhancement device is frictionally engaged with the base at one or more of the plurality of sides and edges of the base. In at least one embodiment, the removable pressure enhancement device is clamped to the strap.

In addition to the foregoing, in at least one embodiment, a tourniquet is provided, comprising: a strap; a windlass operatively associated with the strap; a base interconnected to the strap, the base including an upper surface facing the windlass, a bottom surface, and a plurality of sides and edges; a buckle interconnected to at least one of the strap and the base; means for securing the windlass from rotating; and an occlusion attachment device with a projection having a height of at least 0.5 inch, the occlusion attachment device detachably attached to at least one of: (a) the strap; and (b) the base proximate at least a portion of the bottom surface of the base. In at least one embodiment, the occlusion attachment device is frictionally engaged with the base. In at least one embodiment, the occlusion attachment device is frictionally engaged with one or more of the plurality of sides and edges of the base. In at least one embodiment, the projection comprises a bulbous shape. In at least one embodiment, the occlusion attachment device comprises a clamp.

The one or more present inventions may be embodied in other specific forms without departing from its spirit or essential characteristics. The described embodiments are to be considered in all respects only as illustrative and not restrictive. The scope of the one or more present inventions is, therefore, indicated by the appended claims rather than by the foregoing description. All changes which come within the meaning and range of equivalency of the claims are to be embraced within their scope.

The one or more present inventions, in various embodiments, includes components, methods, processes, systems and apparatus substantially as depicted and described herein, including various embodiments, subcombinations, and subsets thereof. Those of skill in the art will understand how to make and use the one or more present inventions after understanding the present disclosure.

The one or more present inventions, in various embodiments, includes providing devices and processes in the absence of items not depicted and/or described herein or in various embodiments hereof, including in the absence of such items as may have been used in previous devices or processes (e.g., for improving performance, achieving ease and/or reducing cost of implementation).

The foregoing discussion of the one or more present inventions has been presented for purposes of illustration and description. The foregoing is not intended to limit the one or more present inventions to the form or forms disclosed herein. In the foregoing Detailed Description for example, various features of the one or more present inventions are grouped together in one or more embodiments for the purpose of streamlining the disclosure. This method of disclosure is not to be interpreted as reflecting an intention that the claimed one or more present inventions requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the one or more present inventions.

Moreover, though the description of the one or more present inventions has included description of one or more embodiments and certain variations and modifications, other variations and modifications are within the scope of the one or more present inventions (e.g., as may be within the skill and knowledge of those in the art, after understanding the present disclosure). It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An occlusion attachment device for quickly coupling with a tourniquet, the occlusion attachment device comprising:
a bulbous projection attached to a platform, the bulbous projection having a tip located at least 0.5 inch from the platform, the platform further including two oppositely facing surfaces for engaging the tourniquet.

2. The occlusion attachment device of claim 1, wherein the two oppositely facing surfaces include a first surface and an oppositely facing second surface, wherein the first surface of the platform is associated with a first lateral side of the platform, wherein the oppositely facing second surface of the platform is associated with a second lateral side opposite the first lateral side of the platform.

3. The occlusion attachment device of claim 1, wherein the two oppositely facing surfaces include a first surface and an oppositely facing second surface, wherein the first surface of the platform is associated with a floor of the platform.

4. The occlusion attachment device of claim 3, wherein the oppositely facing second surface of the platform is associated with a level opposite the floor of the platform.

5. The occlusion attachment device of claim 4, wherein the level opposite the floor of the platform is interconnected to the floor of the platform by a lateral side of the platform.

6. The occlusion attachment device of claim 4, wherein the level opposite the floor of the platform is interconnected to the floor of the platform by a hinge.

7. The occlusion attachment device of claim 6, wherein the hinge is a living hinge.

8. The occlusion attachment device of claim 1, wherein the bulbous projection is adapted for attaching to the tourniquet without the use of any tools.

9. The occlusion attachment device of claim 1, wherein the bulbous projection comprises a shape selected from the group consisting of a portion of a sphere and a portion of an ellipsoid.

10. An occlusion attachment device for coupling with a tourniquet, the occlusion attachment device comprising:
a platform having a bulbous projection, wherein the platform includes means for quick-connection coupling to the tourniquet.

11. The occlusion attachment device of claim 10, wherein the bulbous projection is adapted for attaching to the tourniquet without the use of any tools.

12. The occlusion attachment device of claim 10, wherein the bulbous projection comprises a shape selected from the group consisting of a portion of a sphere and a portion of an ellipsoid.

13. An assembly for occluding blood flow, comprising:
(a) a tourniquet comprising;
a strap;
a windlass operatively associated with the strap;
a bendable base interconnected to the strap;
a buckle interconnected to at least one of the strap and the bendable base; and
means for securing the windlass from rotating; and
(b) an occlusion attachment device for coupling to the tourniquet, the occlusion attachment device including a bulbous projection, the occlusion attachment device not forming part of the tourniquet;
wherein the tourniquet is operable as a blood occluding device with or without association with the occlusion attachment device.

14. The assembly of claim 13, wherein the occlusion attachment device includes a platform.

15. The assembly of claim 14, wherein the platform includes a first surface and an oppositely facing second surface, and wherein the first surface and the oppositely facing second surface are adapted for engaging a portion of the tourniquet.

16. The assembly of claim 15, wherein the portion of the tourniquet comprises one of the strap and the base.

17. The assembly of claim 15, wherein the first surface of the platform is associated with a first lateral side of the platform.

18. The assembly of claim 17, wherein the oppositely facing second surface of the platform is associated with a second lateral side opposite the first lateral side of the platform.

19. The assembly of claim 15, wherein the first surface of the platform is associated with a floor of the platform.

20. The assembly of claim 19, wherein the oppositely facing second surface of the platform is associated with a level opposite the floor of the platform.

21. The assembly of claim 15, wherein the first surface of the platform is associated with a first jaw member of the platform.

22. The assembly of claim 21, wherein the oppositely facing second surface of the platform is associated with a second jaw member opposite the first jaw member of the platform, and wherein the first jaw member is hingedly attached to the second jaw member.

23. The assembly of claim 13, wherein the occlusion attachment device is adapted for attaching to the tourniquet without the use of any tools.

24. The assembly of claim 13, wherein the bulbous projection comprises a shape selected from the group consisting of a portion of a sphere and a portion of an ellipsoid.

25. The assembly of claim 13, wherein the occlusion attachment device can slide longitudinally along the bendable base of the tourniquet after being attached to the base.
